(19) United States
(12) Patent Application Publication
(10) Pub. No.: US 2026/0151033 A1
(43) Pub. Date: Jun. 4, 2026

(54) WEARABLE FUNDUS EXAMINATION DEVICE

(71) Applicant: National Central University, Taoyuan City (TW)

(72) Inventor: Ching-Cherng SUN, Taoyuan City (TW)

(21) Appl. No.: 19/024,432

(22) Filed: Jan. 16, 2025

(30) Foreign Application Priority Data
Dec. 3, 2024 (TW) 113146723

(51) Int. Cl. A61B 3/12; A61B 3/15; A61B 90/50

(52) U.S. Cl. CPC A61B 3/12; A61B 3/156; A61B 2090/502

(57) ABSTRACT

A wearable fundus examination device includes a main body, mounted on a subject's ear. An imaging module is mounted on the main body and has electrically interconnected an illumination unit, an optical unit, an image capture unit and an external light blocking unit. The illumination unit projects a first light beam and a second light beam. The optical unit guides the first and second light beams to the bottom of the subject's eyeball and generates a reflected light beam. The image capture unit captures the reflected light beam from the bottom of the subject's eyeball converged by the optical unit and form a fundus image. The external light blocking unit is connected to the main body and constructs a dark area between itself and the main body. The dark area covers the subject's eyeball and blocks all visible light, preventing the subject's pupil from constricting by visible light.

### BACKGROUND OF THE INVENTION

### 1. Fields of the invention

The present invention relates to the technical field of fundus examination, and more particularly, to a wearable fundus examination device.

### 2. Descriptions of Related Art

The eyeball is the only place in the body where blood vessels can be observed with the naked eye. Clinically, fundus examination technology is necessary to determine whether subjects have pathological changes, such as retinal detachment, macular disease, diabetes, hypertension, autoimmune diseases, etc. When examining the fundus, most examinations are conducted in hospitals or clinics using testing equipment that fixes the chin of the subject, allowing the subject to undergo fundus imaging examination in sitting or standing positions. However, this examination method can only examine the fundus image when the subject is static and does not include fundus images during postural changes. During postural changes of the subject, the physiological parameters or images of the eyeball will also change accordingly. Furthermore, because everyone's eyeball position is different, the testing equipment needs to have a relatively large range to accurately search for the pupil position, and can only examine one eyeball at a time. The unexamined eyeball will be affected by external light, influencing the line of sight and pupil size of the eyeball being examined. Moreover, the aforementioned fundus examination devices are generally set up in hospitals or health examination institutions and need to be operated by professionals, which is inconvenient for subjects who need long-term monitoring.

The present invention intends to provide a wearable fundus examination device to eliminate the shortcomings mentioned above.

### SUMMARY OF THE INVENTION

The present invention relates to a wearable fundus examination device and comprises a main body mounted on a subject's ear and located corresponding to the subject's eyeball. An imaging module is mounted on the main body and includes electrically interconnected an illumination unit, an optical unit, an image capture unit, and an external light blocking unit. The illumination unit projects a first light beam and a second light beam. The optical unit guides the first light beam and the second light beam to a bottom of the subject's eyeball and generates a reflected light beam. The image capture unit captures the reflected light beam from the bottom of the subject's eyeball converged by the optical unit and form a fundus image. The external light blocking unit is connected to the main body, and a dark area is formed between the external light blocking unit and the main body. The dark area covers the subject's eyeball and blocks all visible light so as to prevent the subject's pupil from constricting due to exposure to visible light.

The primary object of the present invention is to provide a wearable fundus examination device, with the main purpose of performing continuous fundus examination and image capture during subject posture changes without requiring pupil dilators. It is not affected by changes in the subject's posture, and subjects can observe their own fundus images to determine whether any pathological changes have occurred.

The present invention will become more obvious from the following description when taken in connection with the accompanying drawings which show, for purposes of illustration only, a preferred embodiment in accordance with the present invention.

### BRIEF DESCRIPTION OF THE DRAWINGS

FIG. 1 is a schematic diagram of the main body of the present invention in the form of glasses;

FIG. 2 is a circuit block diagram of the present invention, and

FIG. 3 is a schematic diagram showing the relationship between the present invention and the fundus of the eyeball.

### DETAILED DESCRIPTION OF THE PREFERRED EMBODIMENT

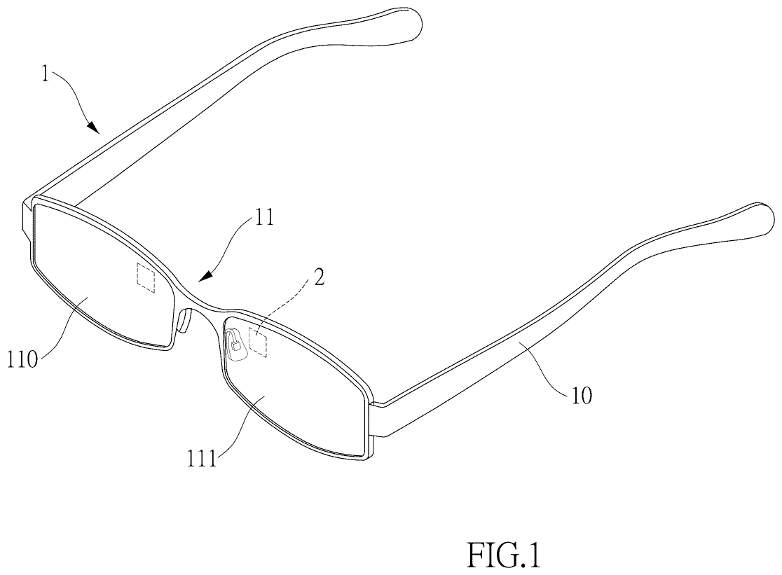

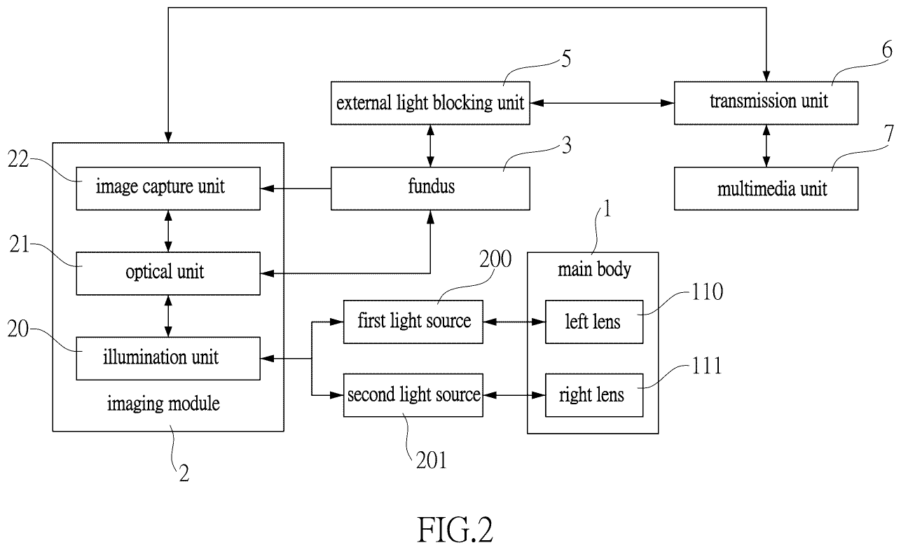

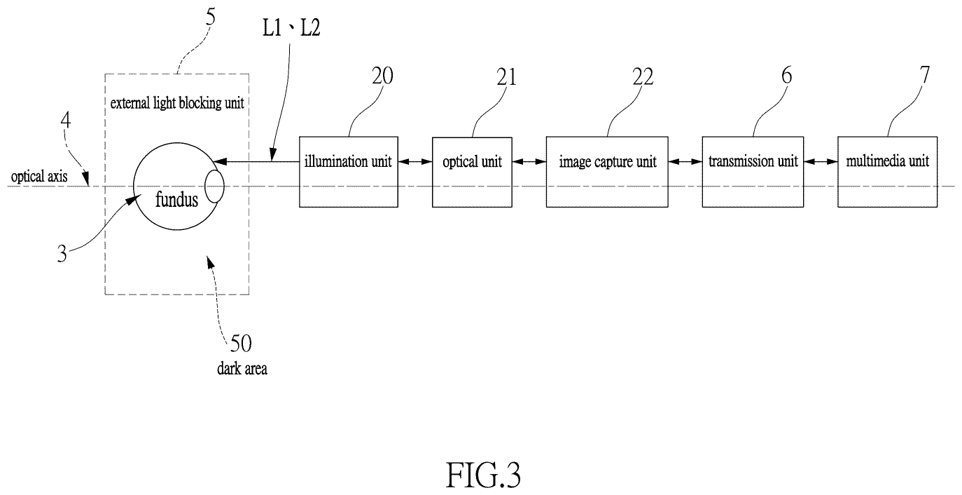

Referring to FIGS. 1 to 3, the wearable fundus examination device of the present invention comprises a main body 1, which can be glasses, an eye mask, or other head-mounted form. In this embodiment, the main body 1 is exemplified by glasses (but not limited to this). The main body 1 has a frame assembly 10 and a lens assembly 11, where the frame assembly 10 is used to position the main body 1 to the subject's ear, and the lens assembly 11 corresponds to the subject's eyeball.

An imaging module 2 is mounted on the lens assembly 11 of the main body 1 and located corresponding to the subject's eyeball. The imaging module 2 has electrically interconnected components including an illumination unit 20, an optical unit 21, and an image capture unit 22.

The illumination unit 20 has a first light source 200 and a second light source 201, wherein the first light source 200 is mounted on the left lens 110 of the lens assembly 11, and the second light source 201 is mounted on the right lens 111 of the lens assembly 11. The first light source 200 and the second light source 201 can be turned on simultaneously or individually depending on the actual application. For example, when examining the subject's left eye, the first light source 200 on the left lens 110 is turned on. When examining the subject's right eye, the second light source 201 on the right lens 111 is turned on. When examining both the subject's left and right eyes simultaneously, both the first light source 200 and the second light source 201 are turned on synchronously.

Furthermore, the first light source 200 and the second light source 201 of the illumination unit 20 project a first light beam L1 and a second light beam L2 respectively. In this embodiment, the first light source 200 and the second light source 201 can use either an Organic Light-Emitting Diode (OLED) or a Liquid-Crystal Display (LCD). The first light beam L1 and second light beam L2 can be different beams, for example, the first light beam L1 is visible light (such as white light), while the second light beam L2 is invisible light (such as infrared).

The optical unit 21 is used to guide the first light beam L1 and the second light beam L2 projected from the illumination unit 20 to the bottom 3 of the subject's eyeball. Furthermore, the optical unit 21 is set between the illumination unit 20 and the image capture unit 22 and matches the object-image relationship with the position of the subject's pupil, allowing it to improve illumination efficiency by adjusting the distance between the illumination unit 20 and the optical axis 4 as well as the optical unit 21. In this embodiment, the optical unit 21 can use either a combination of multiple mirrors or a combination of multiple mirrors and lenses.

The image capture unit 22 is used to capture the reflected light beam from the fundus 3 converged by the optical unit 21 and form a fundus image. In this embodiment, the image capture unit 22 can use either a Charge Coupled Device (CCD) or a Complementary Metal Oxide Semiconductor (CMOS) sensor or a film.

Furthermore, the wearable fundus examination device of the present invention also includes an external light blocking unit 5, a transmission unit 6, and a multimedia unit 7. The external light blocking unit 5 is configured on the main body 1 and constructs a dark area 50 between external light blocking unit 5 and the main body 1. The dark area 50 covers the subject's eyeball and blocks all or substantially all visible light beams that would affect the subject's eyeball, preventing pupil constriction due to the subject receiving visible light beams. The transmission unit 6 is electrically connected to the image capture unit 22, transmitting the fundus image from the image capture unit 22 to the multimedia unit 7 (such as mobile phone, a tablet, or a computer) either wirelessly or through wired connection.

When in use, the subject wears the main body 1, the dark area 50 provided by the external light blocking unit 5 blocks visible light, preventing pupil constriction caused by the subject's pupils receiving visible light. The first light source 200 or the second light source 201 of the optical unit 21 projects visible light (white light) and invisible light (infrared) respectively to the subject's fundus 3. At this time, before the pupil can react and constrict, the image capture unit 22 can clearly capture the subject's fundus image and transmit it through the transmission unit 6 to the subject's multimedia unit 7 (a mobile phone, a tablet, or a computer), allowing the subject to observe their own fundus image and determine whether any pathological changes have occurred.

It is worth mentioning that the dark area 50 provided by the external light blocking unit 5 has the effect of preventing natural pupil dilation, thus eliminating the need to use pupil dilators on the subject. This allows the image capture unit 22 to directly examine and photograph the subject's fundus 3. In addition to effectively shortening the examination time, it also prevents situations where subjects cannot drive or work due to temporary blurred vision caused by pupil dilators.

While we have shown and described the embodiment in accordance with the present invention, it should be clear to those skilled in the art that further embodiments may be made without departing from the scope of the present invention.

What is claimed is:

1. A wearable fundus examination device comprising:
a main body adapted to be mounted on a subject's ear and located corresponding to the subject's eyeball;
an imaging module mounted on the main body and including electrically interconnected an illumination unit, an optical unit, an image capture unit, and an external light blocking unit, the illumination unit projecting a first light beam and a second light beam;
the optical unit guiding the first light beam and the second light beam to a bottom of the subject's eyeball and generating a reflected light beam;
the image capture unit capturing the reflected light beam from the bottom of the subject's eyeball converged by the optical unit and form a fundus image, and
the external light blocking unit connected to the main body, a dark area being formed between the external light blocking unit and the main body, where the dark area covers the subject's eyeball and blocks all visible light so as to prevent the subject's pupil from constricting due to exposure to visible light.

2. The wearable fundus examination device as claimed in claim 1 further comprising a transmission unit, the transmission unit electrically connected to the image capture unit, and transmitting the fundus image from the image capture unit to a multimedia unit through the transmission unit, the multimedia unit including a mobile phone, a tablet, or a computer.

3. The wearable fundus examination device as claimed in claim 1, wherein the illumination unit has a first light source and a second light source, the first light source and the second light source project the first light beam and the second light beam respectively.

4. The wearable fundus examination device as claimed in claim 3, wherein the first light source and the second light source of the illumination unit are either an organic light-emitting diode or a liquid crystal display device.

5. The wearable fundus examination device as claimed in claim 3, wherein the first light beam is a visible light, and the second light beam is an invisible light.

6. The wearable fundus examination device as claimed in claim 3, wherein the first light beam is an invisible light, and the second light beam is a visible light.

7. The wearable fundus examination device as claimed in claim 5, wherein the visible light is a white light, and the invisible light is an infrared light.

8. The wearable fundus examination device as claimed in claim 1, wherein the optical unit is set between the illumination unit and the image capture unit, an illumination efficiency is increased by adjusting a distance between the illumination unit and an optical axis and the optical unit.

9. The wearable fundus examination device as claimed in claim 1, wherein the main body is be a pair of glasses, the glasses have a frame assembly and a lens assembly, the frame assembly positions the main body to the subject's ear, the lens assembly is located corresponding to the subject's eyeball and provides for the mounting of the imaging module.

10. The wearable fundus examination device as claimed in claim 1, wherein the the optical unit is a combination of multiple mirrors and lenses.
